(19) United States
(12) Patent Application Publication
(10) Pub. No.: US 2024/0306682 A1
(43) Pub. Date: Sep. 19, 2024

(54) Process for providing a combination of fermented seaweed and/or algae and plant material selected from the family Fabaceae

(71) Applicant: FERMENTATIONEXPERTS A/S, Bække (DK)

(72) Inventors: Jens Høffner LEGARTH, Bække (DK); Søren KJÆRULFF, Holte (DK)

(21) Appl. No.: 18/036,568

(22) PCT Filed: Nov. 12, 2021
(86) PCT No.: PCT/EP2021/081497

§ 371 Date: May 11, 2023

(30) Foreign Application Priority Data
Nov. 12, 2020 (DK) PA 2020 01271

(51) Int. Cl. A23L 11/50; A23L 5/46; A23L 17/00; A23L 17/60; C12N 1/20

(52) U.S. Cl. CPC A23L 11/50; A23L 5/46; A23L 17/60; A23L 17/65; C12N 1/20

(57) ABSTRACT

The present invention relates to a composition comprising a fermented seaweed and/or a fermented algae; in combination with a fermented plant material, wherein the fermented plant material is selected from the family Fabaceae.

### TECHNICAL FIELD OF THE INVENTION

The present invention relates to a process for providing a combination of fermented seaweed and/or algae and plant material selected from the family Fabaceae. In particular the present invention relates to a process for providing a combination of fermented seaweed (macroalgae) and/or algae (microalgae) and plant material selected from the family Fabaceae where the fermentation process involves a sequential fermentation first comprising the seaweed and/or algae and subsequently involving combination of fermented seaweed and/or algae and plant material selected from the family Fabaceae.

### BACKGROUND OF THE INVENTION

Soybean has for years been used as a source for oil to be used mainly in human food, but also for industrial oils, soaps and biodiesel; and as a source for soybean meal to be used mainly in animal feed as a protein supplement.

Soybean belongs to the scientific classification in the family Fabaceae; and the sub-family Faboideae; within the genus Glycine; and in the species Glycine max(or G. max).

Soybean is a legume native to Asia, widely grown for its consumption either as non-fermented soymilk like tofu, soy nuts, etc.; or as fermented compositions like miso, sufu, natto, etc. Soy-based foods are known to have good nutritional and functional qualities, not only due to their high protein and oil content but also because of phytochemicals, notably the isoflavones.

Acceptance of soybean protein products as animal feed has increased because of low cost and high nutritional value with a good amino acid balance. However, raw soybean may be toxic to non-ruminants due to the presence of high concentration of serine protease inhibitors or trypsin inhibitors.

Processing of soybean produces a wide variety of useful products such as meal, oil, lecithin, and others. Soybean meal is the material remaining after extraction of oil from soybean flakes, with about 48% crude protein content. Apart from being a protein rich product, soybean meal comprises significant amount of anti-nutritional factors and those need to be eliminated to increase its acceptability.

Fermented soybean meal may be produced from soybean meal using fungal, yeast and bacterial strains.

Fermented soybean meal has shown numerous benefits including degradation of soybean allergens during fermentation by microbial proteolytic enzymes. The fermentation process may efficiently eliminate anti-nutritive compounds and improves nutritional value of soybean meal.

Fungal fermentation is the most commons fermentation process used when fermenting soybean meal. When performing bacterial-based fermentation of soybean meal, Bacillus spp. has traditionally been used. For example, the Japanese fermented soybean meal “natto” is made by adding the bacteria B. subtilis to soybean meal. This bacterial fermentation will degrade various anti-nutritional factors of soybean meal including trypsin inhibitors.

Another bacterium used in fermenting soybean meal is Lactobacillus plantarum. Fermentation with lactic acid bacteria, like L. plantarum, has shown to result in protein hydrolysis and increased liberation of free amino acids. Thus, the resulting fermented soybean meal has significantly higher total free amino acid content as compared to soybean meal. The amino acids histidine; threonine; methionine and phenylalanine contents showed no change from fermentation relative to the unfermented soybean meal, the content of leucine; isoleucine; aspartic acid and proline may show to increase from fermentation; whereas amino acids like lysine and alanine shows a decreased content.

One of the challenges with the soymeal products available today is a reduced digestibility of the soymeal protein and thus, the nutritional, functional and pharmacological effects obtained from consuming the soymeal products is reduced.

Thus, there is a continued need in the industry for and new process of providing a soymeal and an improved food and feed products and/or ingredients having improved product characteristics, like improved protein digestibility; without undesirable side effects; and high nutritional, functional and/or pharmacological characteristics and thereby improving the benefits of the food/feed products derived therefrom to humans or animals consuming them.

Hence, an improved process for preparing a fermented soymeal product would be advantageous, and in particular, a more efficient; gentle; and/or controlled process for preparing a fermented soymeal product with improved nutritional, functional and/or pharmacological characteristics would be advantageous.

### SUMMARY OF THE INVENTION

Thus, an object of the present invention relates to an improved process for preparing a soymeal product.

In particular, it is an object of the present invention to provide an improved fermented soymeal product comprising the combination of a seaweed material and/or an algae; and a plant material, with improved nutritional, functional and/or pharmacological characteristics.

Thus, an object of the present invention relates to a composition comprising a fermented seaweed and/or a fermented algae; in combination with a fermented plant material, wherein the fermented plant material is selected from the family Fabaceae.

A further aspect of the present invention relates to a process for providing a composition comprising the combination of fermented seaweeds and/or algae and fermented plant material selected from the family Fabaceae, the process comprises:
a) providing an inoculum comprising (consist essentially of) lactic acid-producing bacteria;
b) providing a first material to be fermented, where said first material comprises (consist essentially of) seaweeds and/or algae;
c) optionally, providing a source of enzyme, such as phosphatases e.g. phytase;
d) combining the materials of steps a), (b) and optionally c), thereby providing a first combinatorial material;
e) fermenting the first combinatorial material of step d) using the inoculum of step a) at a temperature in the range of 15-45° C. and for a period in the range 2-40 days; thereby providing a first fermented composition;
f) combining the first fermented composition of step e) with a second material to be fermented, wherein said second material comprises (consist essentially of) a plant material selected from the family Fabaceae, thereby providing a second combinatorial material;
g) fermenting the second combinatorial material of step f), using the first fermented composition of step e) as a inoculum, at a temperature in the range of 15-45° C. and for a period of at least 2 days, such as at least 3 days, e.g. at least 4 days, such as at least 5 days, such as in the range of 2-15 days, e.g. in the range of 5-12 days, such as 5-10 days, such as 5-7 days or such as 8-10 days; thereby providing a second fermented composition comprising fermented seaweeds and/or algae and fermented plant material selected from the family Fabaceae; and
h) optionally, drying the second fermented composition.

Another aspect of the present invention is to provide a fermented composition obtained/obtainable by the processes according to the invention.

Yet an aspect of the present invention relates to a fermented composition comprising a combination of fermented seaweeds and/or algae and fermented plant material selected from the family Fabaceae which has a protein digestibility of at least 20%.

Still another aspect relates to a food/feed ingredient comprising the fermented composition according to the invention.

A further aspect of the present invention relates to the use of the fermented composition according to the present invention for:
increasing the colour in a salmonoid;
increased growth of fish and/or animals;
reducing mortality;
increased feed conversion; and/or
reduced feed consumption of fish and/or animals.

A further aspect relates to a food/feed product comprising the food/feed ingredient according to the invention.

Still another aspect of the present invention relates to the use of the fermented composition according to the invention in the production of biofuel, such as bio-ethanol and/or antioxidants.

### DETAILED DESCRIPTION OF THE INVENTION

The inventors of the present invention surprisingly found that the method according to the present invention, where fermentation of seaweeds (and/or algae) and plant material selected from the family Fabaceae are combined, a fermented composition may be provided with improved nutritional, functional and/or pharmacological characteristics. The fermented composition (comprising the combination of seaweeds (and/or algae) and plant material selected from the family Fabaceae) showed to have an increased digestibility; increased nitrogen solubility; a reduced excretion of saponins; increased content of small-sized proteins in the range of 35-45 kDa; and/or increased content of lysine; methionine and/or alanine.

Thus, in a preferred embodiment the present invention relates to a process for providing a composition comprising the combination of fermented seaweeds and/or algae and fermented plant material selected from the family Fabaceae, the process comprises:
a) providing an inoculum comprising lactic acid-producing bacteria;
b) providing a first material to be fermented, where said first material comprises seaweeds and/or algae;
c) optionally, providing a source of enzyme, such as phosphatases e.g. phytase;
d) combining the materials of steps a), (b) and optionally c), thereby providing a first combinatorial material;
e) fermenting the first combinatorial material of step d) using the inoculum of step a) at a temperature in the range of 15-45° C. and for a period in the range 2-40 days; thereby providing a first fermented composition;
f) combining the first fermented composition of step e) with a second material to be fermented, wherein said second material comprises a plant material selected from the family Fabaceae, thereby providing a second combinatorial material;
g) fermenting the second combinatorial material of step f), using the first fermented composition of step e) as a inoculum, at a temperature in the range of 15-45° C. and for a period of at least 2 days, such as at least 3 days, e.g. at least 4 days, such as at least 5 days, such as in the range of 2-15 days, e.g. in the range of 5-12 days, such as 5-10 days, such as 5-7 days or such as 8-10 days; thereby providing a second fermented composition comprising fermented seaweeds and/or algae and fermented plant material selected from the family Fabaceae; and
h) optionally, drying the second fermented composition.

In the contest of the present invention the term “fermented seaweeds and/or algae and fermented plant material” relates to the combination of a seaweed and a plant material; the combination of an algae and a plant material; or the combination of a seaweed and an algae and a plant material. In a preferred embodiment of the present invention the term “fermented seaweeds and/or algae and fermented plant material” relates to the combination of a seaweed and a plant material.

In another preferred embodiment the present invention relates to a process for providing a composition comprising the combination of fermented seaweeds and/or algae and fermented plant material selected from the family Fabaceae, the process comprises:
a) providing an inoculum consist essentially of lactic acid-producing bacteria;
b) providing a first material to be fermented, where said first material consist essentially of seaweeds and/or algae;
c) optionally, providing a source of enzyme, such as phosphatases e.g. phytase;
d) combining the materials of steps a), (b) and optionally c), thereby providing a first combinatorial material;
e) fermenting the first combinatorial material of step d) using the inoculum of step a) at a temperature in the range of 15-45° C. and for a period in the range 2-40 days; thereby providing a first fermented composition;
f) combining the first fermented composition of step e) with a second material to be fermented, wherein said second material comprises consist essentially of a plant material selected from the family Fabaceae, thereby providing a second combinatorial material;
g) fermenting the second combinatorial material of step f), using the first fermented composition of step e) as a inoculum, at a temperature in the range of 15-45° C. and for a period of at least 2 days, such as at least 3 days, e.g. at least 4 days, such as at least 5 days, such as in the range of 2-15 days, e.g. in the range of 5-12 days, such as 5-10 days, such as 5-7 days or such as 8-10 days; thereby providing a second fermented composition comprising fermented seaweeds and/or algae and fermented plant material selected from the family Fabaceae; and
h) optionally, drying the second fermented composition.

In the context of the present invention, the term “comprising” relates to the interpretation of a feature as meaning that this/those feature(s) are included, but it does not exclude the presence of other features.

In the context of the present invention, the term “consisting essentially of”, relates to a limitation of the scope of a claim to the specified features or steps and those features or steps, not mentioned and that do not materially affect the basic and novel characteristic(s) of the claimed invention.

In an embodiment of the present invention a further inoculum comprising lactic acid-producing bacteria is added to the second combinational material provided in step (f). In a further embodiment of the present invention a further inoculum consisting essentially of lactic acid-producing bacteria is added to the second combinational material provided in step (f)

Preferably, the content of the further inoculum as provided in step (f) may be the same as the inoculum as provided in step (a).

The inventors of the present invention surprisingly found that by making the combined, but sequential fermentation process of seaweed and/or algae and plant material selected from the family Fabaceae a fermented composition may be produced which has:
increased digestibility;
increased protein dispersibility;
increased nitrogen solubility;
reduced excretion of saponins;
increased content of small-sized proteins in the range of 35-45 kDa; increased amino acid content, in particular the content of lysine; methionine and/or alanine; and/or
reduced content of lectins and trypsin inhibitors that impairs protein digestion;

Preferably the above-mentioned improvements to the fermented composition may be provided at the same time as the fermented composition may comprise an improved nutritional, functional and/or pharmacological characteristics.

In an embodiment of the present invention the fermented composition produced may have an increased digestibility relative to a plant material selected from the family Fabaceae but which has not been fermented and/or relative to a plant material selected from the family Fabaceae but which has been fermented without the co-fermentation with seaweed and/or algae.

In the present context the term “co-fermentation with seaweed and/or algae” relates to the fermentation process according to the present invention, where the seaweed and/or the algae is initially fermented followed by addition of the plant material selected from the family Fabaceae, whereby the fermentation is continued resulting in the fermented composition according to the present invention.

In a further embodiment of the present invention the fermented composition produced according to the present invention may have a digestibility, such as an amino acid digestibility, of 20% or more; such as 30% or more; e.g. 40% or more; such as 50% or more; e.g. 65% or more; such as 75% or more; e.g. 85% or more; such as 87% or more; e.g. 89% or more; such as 90% or more; e.g. 91% or more; such as 93% or more; e.g. 95% or more; such as 97% or more; e.g. 99% or more.

The digestibility of the fermented composition produced according to the present invention may preferably be a true ileal digestibility. Preferably, the digestibility, e.g. the true ileal digestibility, is determined by the method described by H. H. Stein et al .; Livestock Science, Volume 109, Issues 1-3, 15 May 2007, Pages 282-285, “Definition of apparent, true, and standardized ileal digestibility of amino acids in pigs”

The fermented composition produced according to the present invention may have an increased nitrogen solubility relative to a plant material selected from the family Fabaceae but which has not been fermented and/or relative to a plant material selected from the family Fabaceae but which has been fermented without the co-fermentation with seaweed and/or algae.

In the present context the terms “nitrogen solubility” and “nitrogen solubility index” relates to a percentage of the protein nitrogen that is water soluble relative to the total protein nitrogen present.

In a further embodiment of the present invention the fermented composition produced according to the present invention may have a nitrogen solubility of 65% or more; such as 70% or more; e.g. 75% or more; such as 80% or more; e.g. 85% or more; such as 90% or more; e.g. 92% or more; such as 94% or more; e.g. 95% or more.

The nitrogen solubility of the fermented composition produced according to the present invention may preferably be a KOH-nitrogen solubility. Preferably, the nitrogen solubility, e.g. the KOH-nitrogen solubility, is determined by the method described by Caprita R. et. al./Scientific Papers: Animal Science and Biotechnologies, 2010, 43 (1).

The fermented composition produced according to the present invention may have an increased protein dispersibility relative to a plant material selected from the family Fabaceae but which has not been fermented and/or relative to a plant material selected from the family Fabaceae but which has been fermented without the co-fermentation with seaweed and/or algae.

In the present context the term “protein dispersibility” relates to means of comparing the solubility of a protein in water. The protein dispersibility is determined by grinding a sample of the fermented composition produced according to the present invention and mixing with a specific quantity of water and blending at a specific rpm for a specific time. The resulting mixture and comparing soymeal have the protein contents measured using a combustion test. The protein dispersibility is calculated as the percentage of the protein in the mix divided by the percentage in the comparing soymeal.

In a further embodiment of the present invention the fermented composition produced according to the present invention may have a protein dispersibility of 32% or more; such as 35% or more; e.g. 38% or more; such as 40% or more; e.g. 45% or more; such as 50% or more; e.g. 55% or more; such as 60% or more; e.g. 65% or more.

The fermented composition produced according to the present invention may have an increased content of small-sized proteins in the range of 35-45 kDa, relative to a plant material selected from the family Fabaceae but which has not been fermented and/or relative to a plant material selected from the family Fabaceae but which has been fermented without the co-fermentation with seaweed and/or algae.

In an embodiment of the present invention the fermented composition produced according to the present invention may have an increased content of small-sized proteins in the range of 35-45 kDa of at least 10% relative to a plant material selected from the family Fabaceae but which has not been fermented and/or relative to a plant material selected from the family Fabaceae but which has been fermented without the co-fermentation with seaweed and/or algae; such as at least 20%; e.g. at least 25%; such as at least 30%; e.g. at least 35%; such as at least 40%; e.g. at least 45%; such as at least 50%; e.g. at least 75%; such as at least 90%; e.g. at least 100%.

The increased content of peptides in the range of 35-45 kDa may be determined by SDS-PAGE (sodium dodecyl sulfate-polyacrylamide gel electrophoresis) or by mass spec.

In an embodiment of the present invention the small-sized proteins are in the range of 35-45 kDa; such as in the range of 36-42 kDa; e.g. in the range of 37-39 kDa.

The fermented composition produced according to the present invention may have an increased content of lysine; methionine and/or alanine relative to a plant material selected from the family Fabaceae but which has not been fermented and/or relative to a plant material selected from the family Fabaceae but which has been fermented without the co-fermentation with seaweed and/or algae.

In the present context the term “increased content of lysine; methionine and alanine” relates to an increased context of either lysine; methionine or alanine; or a combination of lysine and methionine; lysine and alanine; methionine and alanine; or lysine and methionine and alanine.

In an embodiment of the present invention the increased content of lysine; methionine and alanine is an increase in free lysine; methionine and/or alanine.

In a further embodiment of the present invention the fermented composition produced according to the present invention comprise an amino acid content, preferably a free amino acid content, of at least 2% higher than the amino acid content, preferably the free amino acid content, of a plant material selected from the family Fabaceae but which has not been fermented and/or a plant material selected from the family Fabaceae but which has been fermented without the co-fermentation with seaweed and/or algae; such as at least 4%; e.g. at least 6%; such as at least 8%; e.g. at least 10%; such as at least 12%; e.g. at least 15%; such as at least 17%; e.g. at least 20%; such as at least 22%; e.g. at least 25%; such as at least 27%; e.g. at least 30%.

In yet an embodiment of the present invention the fermented composition produced according to the present invention comprise a lysine content, preferably a free lysine content, of at least 2% higher than the lysine content, preferably the free lysine content, of a plant material selected from the family Fabaceae but which has not been fermented and/or a plant material selected from the family Fabaceae but which has been fermented without the co-fermentation with seaweed and/or algae; such as at least 4%; e.g. at least 6%; such as at least 8%; e.g. at least 10%; such as at least 12%; e.g. at least 15%; such as at least 17%; e.g. at least 20%; such as at least 22%; e.g. at least 25%; such as at least 27%; e.g. at least 30%.

In a further embodiment of the present invention the fermented composition produced according to the present invention comprise a methionine content, preferably a free methionine content, of at least 1% higher than the methionine content, or the free methionine content, of a plant material selected from the family Fabaceae but which has not been fermented and/or relative to a plant material selected from the family Fabaceae but which has been fermented without the co-fermentation with seaweed and/or algae; such as at least 2%; e.g. at least 3%; such as at least 5%; e.g. at least 7%; such as at least 10%.

In an even further embodiment of the present invention the fermented composition produced according to the present invention comprise an alanine content, preferably a free alanine content, of at least 5% higher than the alanine content, preferably the free alanine content, of a plant material selected from the family Fabaceae but which has not been fermented and/or a plant material selected from the family Fabaceae but which has been fermented without the co-fermentation with seaweed and/or algae; such as at least 10%; e.g. at least 15%; such as at least 20%; e.g. at least 25%; such as at least 30%; e.g. at least 35%; such as at least 40%; e.g. at least 50%; such as at least 55%; e.g. at least 60%; such as at least 65%; e.g. at least 70%.

Few compounds in plant materials selected from the family Fabaceae, such as soybeans, are found to impair protein digestion. These compounds are naturally present in the plant as a result of an evolutionary trait. The two most important are lectins and trypsin inhibitors. Lectins are proteins that have a high affinity for binding to carbohydrates, creating complex structures that are difficult to digest. Lectins also bind to the gut mucosa prompting an inflammation reaction. The second ones are trypsin inhibitors. These are small peptides characterized by their ability to bind to trypsin and chymotrypsin proteases. Trypsin is the most important pancreatic enzyme which role is to mature all other pancreatic enzymes but also to break down protein at the small intestine.

The level of lectins and trypsin inhibitors, in particular lectins and trypsin inhibitors that are found to impair protein digestion, are preferably reduced during fermentation according to the present invention. In an embodiment of the present invention, the process, e.g. the fermentation process, according to the present invention does not involve a specific step and/or action of inactivating lectins and trypsin inhibitors found to impair protein digestion.

In an embodiment of the present invention, inactivation of lectins and trypsin inhibitors that are found to impair protein digestion may involve heating, chemical treatment or enzymatic treatment of the plant material selected from the family Fabaceae.

Another undesirably compound of the present invention is saponins. Saponins are complex molecules found in many plants including plant materials selected from the family Fabaceae, such as soybeans. Saponins are formed by a carbohydrate chain and steroids, steroid alkaloids or triterpenes. When dissolved in water they form a stable soapy froth. Because of the presence of both a hydrophilic (sugar) and hydrophobic (triterpenes and steroids) properties, they act as emulsifiers and foaming agents.

In an embodiment of the present invention, the fermented composition obtained by the process according to the present invention may have a reduced content of saponins relative to a plant material selected from the family Fabaceae but which has not been fermented and/or relative to a plant material selected from the family Fabaceae but which has been fermented without the co-fermentation with seaweed and/or algae. Preferably, the reduction in the content of saponins in the fermented composition obtained by the process according to the present invention involves at least a 5% (w/w) reduction; such as a 10% (w/w) reduction; e.g. a 15% (w/w) reduction; such as a 20% (w/w) reduction; e.g. a 25% (w/w) reduction; such as a 30% (w/w) reduction; e.g. a 35% (w/w) reduction; such as a 40% (w/w) reduction; e.g. a 45% (w/w) reduction.

By using the partly fermented seaweed and/or algae material as an inoculum for fermentation of a plant material selected from the family Fabaceae, a final product may be provided with beneficial properties. It has a high protein content constituted of a mixture from several sources and a high digestibility due to the combined fermentation. The required prolonged fermentation period for algae/seaweed may be used in a time-optimized way, such that at the end of the process, both the plant material and the seaweed/algae material may be fermented for a required period of time and temperature.

In addition, the beneficial antioxidants, peptides and/or proteins may also be made available by the sequential fermentation and prolonged fermentation of the seaweed/algae. This may benefit the improved nutritional, functional and/or pharmacological characteristics of the fermented composition obtained by the process of the present invention.

“Inoculation” refers the placement of a microorganism(s) (e.g. lactic acid producing bacteria) that will grow when implanted in a culture medium such as a fermentation tank comprising media to be fermented. “Inoculum” refers to the material used in an inoculation, for example a composition comprising a living organism(s), which is employed to prime a process of interest. For example, an inoculum where the bacteria are essentially consisting of lactic acid producing bacteria may be used to direct a lactic acid formation process in a culture medium in a fermentation tank comprising said media (e.g. seaweeds and/or algae and plant material selected from the family Fabaceae). Thus, “to inoculate” refers to the transfer of the inoculum to the media to be processed, for example the transfer of the inoculums to the seaweeds and/or algae or the fermented plant material selected from the family Fabaceae to be fermented; optionally, in combination with a source of phytase.

The inoculation may be a fermented feed product which comprises viable lactic acid producing bacteria in sufficient amount to prime a lactic acid fermentation process of another feed product (e.g. the seaweeds and/or algae or the plant material selected from the family Fabaceae) to be fermented.

The inoculum may be a in a liquid form, dry form, or essentially dry form.

The moisture % of the inoculum may be adjusted in order to optimize the fermentation process. Thus, the inoculum used in the processes of the present invention may be a fermented feed product. In one embodiment the inoculum is provided as essentially pure viable bacteria (such as bacteria in freeze dried form) or bacteria suspended in a suitable media prior to the application (such as a water, buffer or a growth media).

The proportion of the inoculums added to the feed product comprising said protein supplement may vary. In case it is considered that the load of undesirable microbes are significant in the feed product or the fermentation system, the proportion of the inoculum in the fermentation mixture (inoculum +feed product comprising protein supplement +additional water) may be increased to insure that the fermentation is directed by the microbes (e.g. by the lactic acid bacteria) of the inoculums.

In an embodiment of the present invention, the inoculum may be provided with a concentration of lactic acid bacteria in the inoculum sufficient to outgrow any bacteria, yeast or moulds present in the first material of step (b) and/or in the second material of step (f).

Accordingly, in one embodiment of the invention, the proportion of said inoculum in the combined materials provided in step (d) and/or the proportion of inoculum in the second combinatorial material of step (f), may be in the range of 0.1 to 99.9 vol-%, 1 to 99 vol-%, 5 to 70 vol-%, 10 to 50 vol-%, or 25 to 35 vol-%, 0.1-10 vol-%, or 0.5-5 vol-%, or 1-2.5 vol-%, or around 1-2 vol-vol-%.

The lactic acid bacteria comprise a clade of Gram positive, low-GC, acid tolerant, non-sporulating, non-respiring rod or cocci that are associated by their common metabolic and physiological characteristics. These bacteria usually found in decomposing plants cells and dairy products produce lactic acid as the major metabolic end-product of carbohydrate fermentation. This trait has historically linked lactic acid bacteria with food fermentations as acidification inhibits the growth of spoilage agents. Proteinaceous bacteriocins are produced by several lactic acid bacteria strains and provide an additional hurdle for spoilage and pathogenic microorganisms.

Furthermore, the fermentation process according to the present invention may provide lactic acid and other metabolic products which contribute to improved organoleptic and textural profile of fermented composition and the food or feed product according to the present invention.

The industrial importance of the lactic acid bacteria is further evidenced by their generally regarded as safe (GRAS) status, due to their ubiquitous appearance in food and their contribution to the healthy microflora of human mucosal surfaces.

In the present invention, the lactic acid-producing bacteria in inoculum used for fermentation may preferably be lactic acid bacteria of the genus Enterococcus, Lactobacillus, Pediococcus or Lactococcus, or combinations thereof.

In an embodiment of the present invention the inoculum comprises at least one lactic acid bacterium species selected from the group consisting of one or more of Enterococcus spp., Lactobacillus spp., Lactococcus spp., and Pediococcus spp.

In yet an embodiment of the invention, the lactic acid bacteria may be selected from the group consisting of one or more of Enterococcus faecium, Lactobacillus rhamnosus, Lactobacillus plantarum, Pediococcus acidililactili, and Pediococcus pentosaceus.

In further embodiment, the lactic acid producing bacteria may be of the order Lactobacillies. The lactic acid-producing bacteria can also be selected from Lactobacillus spp., Pediococcus spp., Enterococcus spp., and Lactococcus spp. or a combination thereof.

In another embodiment, the lactic acid-producing bacteria comprise Pediococcus pentosaceus, Pendiococcus acidilactici and Lactobacillus plantarum, Lactobacillus rhamnosus, and Enterococcus faecium, or a combination thereof.

In still another embodiment, the lactic acid bacteria comprise Enterococcus faecium and/or Lactobacillus rhamnosus.

In yet a further embodiment of the present invention, the lactic acid bacteria comprise one or more of Enterococcus faecium MCIMB 30122, Lactobacillus rhamnosus NCIMB 30121, Pediococcus pentosaceus HTS (LMG P-22549), Pendiococcus acidilactici NCIMB 30086 and/or Lactobacillus plantarum LSI (NCIMB 30083).

In a further embodiment of the invention, the inoculum of step (a) has been obtained by fermentation with primary inoculum comprising at least one lactic acid bacterium species selected from the group consisting of one or more of Enterococcus spp., Lactobacillus spp., Lactococcus spp., and Pediococcus spp.

The inoculum of the present invention may be a combination of lactic acid bacteria and industrial by-products.

A by-product according to the present invention may a material, which is not the main product of a production process, but a product, which may find use in other processes. An example of a by-product may be potato peels.

Thus, in an embodiment the inoculum in step a) comprises a mixture of lactic acid bacteria and organic by-products of an industrial process. In yet another embodiment the by-product is potato peels.

In a further embodiment the lactic acid bacteria and organic by-products have been mixed directly after the by-products have been generated, thereby minimizing unwanted bacterial contamination. To save energy during the entire process an embodiment of the invention relates to a process wherein no sterilization of the by-products have taken place after the by-products have been generated.

By mixing the lactic acid bacteria with the by-product directly after the by-product has been generated unwanted contamination is avoided.

In the present context, the term “by-product” refers to products deriving from industrial processes, which may be available at low costs, or for free. Commonly, they are not used directly as feed for animals, and long-time storage may be an issue due to decomposition and uncontrolled fermentation and spoilage. Examples of such “by-products” are whey, spent grain (from brewing, wine or bio-ethanol industry), plant or parts thereof, potatoes, and potato peels.

In a preferred embodiment the by-product(s) have been sterilized during the processing of the main product, thereby making subsequent sterilization steps an unnecessary requirement.

In the present context “sterilization” refers to any process that eliminates (removes) or kills essentially all forms of microbial life, including transmissible agents (such as fungi, bacteria, viruses, spore forms, etc.) present on a surface or in the product. Sterilization may be achieved by applying the proper combinations of heat, chemicals, irradiation, high pressure, and filtration.

In an embodiment of the present invention potato peeling at industrial scale the potato peel is loosened from the potato before peeling by steaming. Thus, by mixing the potato peels (by-product) with the lactic acid bacteria directly after the potato peels have been removed from the complete potato no further sterilisation may be required later on in the process according to the invention.

By “directly after” it is to be understood as within a period, which does not allow e.g. a bacterial fauna of unwanted origin to be established before the lactic bacteria is added to the by-product.

In heterolactic acid fermentation, one molecule of pyruvate is converted to lactate; the other is converted to ethanol and carbon dioxide. In homolactic acid fermentation, both molecules of pyruvate are converted to lactate. Homolactic acid fermentation is unique because it is one of the only respiration processes to not produce a gas as a byproduct.

Homolactic fermentation breaks down the pyruvate into lactate. It occurs in the muscles of animals when they need energy faster than the blood can supply oxygen. It also occurs in some kinds of bacteria (such as lactobacilli). It is this type of bacteria that converts lactose into lactic acid in yogurt, giving it its sour taste. These lactic acid bacteria can be classed as homofermentative, where the end product is mostly lactate, or heterofermentative, where some lactate is further metabolized and results in carbon dioxide, acetate or other metabolic products.

In homolactic fermentation, one molecule of glucose is converted to two molecules of lactic acid. In heterolactic fermentation one molecule of glucose being converted to one molecule of lactic acid, one molecule of ethanol, and one molecule of carbon dioxide

The lactic acid bacteria according to the invention may be capable of producing lactic acid and decrease the pH during fermentation to 4.2 or below within 24 hours; such as within 20 hours; e.g. within 15 hours; such as within 12 hours; e.g. within 10 hours.

It is preferred that the fermented composition of the present invention is obtained by lactic acid fermentation. It is also preferred that the fermentation is homolactic fermentation directed by homofermentative lactic acid bacteria. In one embodiment, the fermentation is heterolactic fermentation.

The type of seaweed and/or algae may be selected from numerous types of seaweed. In the present context the term “seaweed” encompasses macroscopic, multicellular, benthic marine algae. The term includes members of the red, brown and green algae. Algae are a very large and diverse group of simple, typically autotrophic organisms, ranging from unicellular to multicellular forms, such as the giant kelps that grow to 65 meters in length. Most are photosynthetic and “simple” because they lack the many distinct cell and organ types found in land plants. The largest and most complex marine forms are called seaweeds.

In an embodiment of the present invention the seaweed/algae provided in step b) may be selected from the group consisting of red, brown and/or green algae. In yet an embodiment the seaweed may be selected from the group consisting of Laminaria saccharina (sugar kelp); Laminaria digitata; Laminaria hyperborean; gracilaria; Saccharina latissimi; and Ascophyllum nodosum.

Phytase (myo-inositol hexakisphosphate phosphohydrolase) is a type of phosphatase enzyme that catalyzes the hydrolysis of phytic acid (myo-inositol hexakisphosphate), an undigestable, organic form of phosphorus present in for example grains. A useable form of inorganic phosphorus is released by the hydrolysis of phytic acid. Thus, phytase may increase phosphor uptake when the final composition is used in a feed product and/or a food product.

In an embodiment, the first and/or the second combinatorial product comprises a source of phytase in the form of grain or bran, such as wheat and/or triticale bran. In yet an embodiment the content of said source of phytase may be in the range 1-40% by weight, such as 10 to 40% by weight, such as in the range of 10 to 25% by weight, such in the range of 15 to 20% by weight.

In an embodiment of the present invention the plant material selected from the family plant material selected from the family Fabaceae may be selected from the groups consisting of Glycine max (soybean); Phaseolus (beans); Pisum sativum(pea); Vicia faba (broad bean, fava bean, or faba bean); Cicer arietinum (chickpeas); Medicago sativa (alfalfa); Arachis hypogaea (peanut); Ceratonia siliqua (carob); and Glycyrrhiza glabra (liquorice).

The Glycine max may be selected from soybean or a fraction hereof, preferably the fraction of soybean may be soya cake.

In an embodiment of the present invention the plant material may in addition to the plant material selected from the family plant material selected from the family Fabaceae, comprise a second plant material. The second plant material may be preferably be a plant material different from the family plant material selected from the family Fabaceae. Preferably, the second plant material may be selected for the family of Brassicaceae.

In an embodiment of the present invention the Brassicaceae family may be selected from at least one of a Brassica genus; sun flower; palm; soya, field beans, Lupins; or a combination hereof. Preferably, at least one Brassica genus may be selected from one or more species such as Brassica napus; Brassica oleracea; Brassica campestris; Brassica nigra; Sinapis alba (Brassica alba); Brassica juncea; Brassica rapa or mixtures hereof.

In yet an embodiment of the present invention the at least one Brassica genus may be selected from the group consisting of: including rape, rapeseed, canola, cabbage, broccoli, cauliflower, kale, Brussels sprouts, collard greens, savoy, kohlrabi, gai lan, white mustard, Indian mustard, Chinese mustard, and black mustard seed powder.

In an embodiment of the present invention the composition according to the present invention may comprise ratio of the fermented plant material between:
(i) the plant material selected from the family Fabaceae; and
(ii) the second plant material;

Being at least 50/50; such as at least 60/40, e.g. at least 70/30; e.g. at least 80/20; such as at least 90/10 (the plant material selected from the family Fabaceae/the second plant material).

In an embodiment of the present invention the fermented plant material of the composition comprises at least 60% of the plant material selected from the family Fabaceae; such as at least 70%; e.g. at least 80%; such as at least 90%; e.g. at least 92%; such as at least 94%; e.g. at least 96%; such as at least 98%; e.g. 100%.

In a further embodiment of the present invention the fermented plant material of the composition comprises at most 40% of the second plant material; such as at most 30%; e.g. at most 20%; such as at most 10%; e.g. at most 8%; such as at most 6%; e.g. at most 4%; such as at most 2%.

In yet an embodiment of the present invention the fermented plant material of the composition comprises at least 60% of the plant material selected from the family Fabaceae; such as at least 70%; e.g. at least 80%; such as at least 90%; e.g. at least 92%; such as at least 94%; e.g. at least 96%; such as at least 98%; and the fermented plant material of the composition comprises at most 40% of the second plant material; such as at most 30%; e.g. at most 20%; such as at most 10%; e.g. at most 8%; such as at most 6%; e.g. at most 4%; such as at most 2%. Preferably the fermented plant material of the composition comprises at least 80% of the plant material selected from the family Fabaceae and at most 20% of the second plant material, such as at least 90% of the plant material selected from the family Fabaceae and at most 10% of the second plant material.

In a further embodiment of the present invention neither the seaweeds; the algae or the plant material selected from the family Fabaceae, is subjected to a sterilization step. Preferably, the seaweeds; the algae or the plant material selected from the family Fabaceae is not subjected to temperatures above 95° C.; such as temperatures above 90°° C.; e.g. temperatures above 80° C.; such as temperatures above 75° C.

The fermentation process in step e) may preferably be controlled by varying the temperature and time of the fermentation process to optimize the fermentation reaction.

Thus, in an embodiment of the present invention the fermentation of the first combinatorial material of step e) and/or the fermentation of the second combinatorial material of step g) is performed at a temperature in the range 15-45°° C., such as 15-40° C., such as 25-35°° C., such as 30-40°° C., such as 15-20° C. or such as 40-45° C.

In a further embodiment of the present invention the fermentation of the first combinatorial material of step e) is performed for a period in the range 2-40 days, such as 5-40 days, such as 10-14 days, such as 15-40 days, such as 20-40 days, such as 25-40 days, such as 30-40 days, preferably for at least 15 days, such as at least 30 days, such as at least 100 days, or such as at least 200 days.

In a further embodiment of the present invention the fermentation of the second combinatorial material of step g) is performed for a period of 2 days, such as at least 3 days, e.g. at least 4 days, such as at least 5 days, such as in the range of 2-15 days, e.g. in the range of 5-12 days, such as 5-10 days, such as 5-7 days or such as 8-10 days.

When the fermentation process runs for longer periods (such as more than 6 days; e.g. more than 8 days; such as more than 10 days; e.g. more than 12 days), the microbial activity may decrease or cease completely due to lowered viability of the bacteria. However, since enzymatic degradation may continue, it may be advantageous to continue the process. Furthermore, since the pH has been lowered during fermentation, contamination from undesired micro-organisms may be minimized.

In an embodiment of the present invention a further inoculum consisting essentially of lactic acid-producing bacteria may be added to the second combinational material provided in step (f). Preferably, the content of the further inoculum as provided in step (f) may be the same as the inoculum as provided in step (a).

The compositions of the invention may be dried if the optional drying step of the processes of the invention is employed. In the present context a dry product is to be understood as a product having a water content of 16% (w/w) or less; a water content not exceeding 18% (w/w); such as not exceeding 14% by weight dry matter; e.g. 14% by weight dry matter.

The moisture content of the fermentation step e) may vary. Since seaweeds have a natural high water content directly after harvesting, it is important that the process can run efficiently at high water content. Thus, in yet an embodiment the moisture content during the fermentation step e) is in the range 25-85%, such as in the range 27.5% to 50%, preferably 32 to 38% by weight dry matter (wt %).

In certain instances, it may be advantageous to lower the water content of the seaweeds before the fermentation step e) is initiated. Thus, in an embodiment the moisture content of the seaweeds is lowered to a moisture content below 70% before step e) or during step b), such as to a moisture content below 60%, such as to a moisture content below 50%, such as to a moisture content below 40%, such as to a moisture content in the range 10-40%, such as in the range 20-40%, such as 30-40%. In yet an embodiment the water content is lowered by mechanical means, such as a screw press. In another embodiment the moisture content is lowered by drying, such as by exposure to the sun.

To increase the surface area of the seaweeds during the fermentation step it may also be advantageous to process the seaweeds. Thus, in an especially preferred embodiment the seaweeds and/or algae; or the plant material selected from the family Fabaceae are grinded, cut, chopped, sliced, and/or fractionized before or during fermentation. In a specific embodiment the fractionized seaweeds and/or algae; or the plant material selected from the family Fabaceae have an average maximum diameter of 5 cm, such as an average maximum diameter of 4 cm such as an average maximum diameter of 3 cm, such as an average maximum diameter of 2 cm, such as an average maximum diameter of 1 cm, such as an average diameter in the range 25 μm to 5 cm, such as 0.1 mm to 5 cm, such as an average diameter in the range of 0.5 mm to 5 cm, such as an average diameter in the range 0.5 mm to 2 cm.

In an embodiment of the present invention the amount of seaweed and/or algae by weight dry matter of the first combinatorial material in step (d) may vary. Thus, in an embodiment the seaweed and/algae material constitutes more than 10% by weight dry matter (wt %) of the first combinatorial material in step d), such as more than 17 wt %, such as more than 20 wt %, such more than 22 wt %, such as more than 24 wt %, such as more than 26 wt % such as more than 28 wt % such as more than 30 wt %, such as more than 35 wt %, such as more than 40 wt %, or such as more than 50%, e.g. 10-55%; such as 15-25%. It may be advantageous quickly to add the inoculum to the seaweed and/or algae in step (d) in order to avoid contamination of the harvested seaweeds/algae by undesired micro-organisms. This may be avoided by adding inoculum comprising lactic acid producing bacteria instantly or almost instantly to the harvested seaweeds/algae. In an embodiment the inoculum comprising lactic acid producing bacteria may be added to the harvested seaweed and/or algae within 7 days after harvesting, such as within 6 days, such as within 5 days, such as within 4 days, such as within 3 days such as within 2 days, such as within 1 day, such as within 12 hours, such as within 6 hours, such as 4 hours, such as 2 hours, such as 1 hour such as 30 minutes, such as 10 minutes after harvesting. Preferably the inoculum is added instantly to the harvested seaweeds and/or algae.

In another embodiment the harvested seaweed is conserved before an inoculum may be added. In such instances the harvested seaweed may be stored at least days, such as at least 40 days, such as at least 60 days such as at least 100 days, such as in the range 20-300 days, or such as in the range 20-100 days.

By addition of the lactic acid producing bacteria the pH in the composition is lowered to a pH of 4.2 or less, such as a pH of 4.0 or less; e.g. a pH in the range of 3.5-4.2; such as a pH in the range of 3.5 or 3.8 due to the controlled fermentation. At these low pH's the risk of contamination from other micro-organisms may be limited since they cannot divide under these conditions.

Thus, in one embodiment of the invention, the pH of inoculum is below 4.2. In another embodiment, the pH of the inoculum of step (a) is 4.2 or below, such as in the range of 4.2-3.5, in the range of 3.9-3.7, or 3.8.

In one embodiment, the pH is lowered to a pH in the range 3.5 to 4.2 during fermentation step e) such as to around 3.8.

Since seaweed farms of natural reasons often are positioned off-shore it would be advantageous also to be able to perform the fermentation off-shore, since it would otherwise be difficult to initiate the fermentation process before the harvested seaweed/algae starts to decompose due to contamination of undesired micro-organisms. Thus, in an embodiment at least step a) to e) of said process is carried out off-shore, such as on a ship, on a barge, or on a on an offshore platform. It is to be understood that the fermentation process may continue on-shore until a drying process may be initiated. Alternatively, the ship or barge may be emptied directly into a dryer positioned on a harbour. In this way transport of heavy wet fermented compositions may be avoided. In yet an embodiment the fermented seaweed may be emptied into a second fermentation area/chamber, wherein the fermented seaweed is added to (and mixed in) the further proteinaceous plant materials to be fermented (as outlined above).

As mentioned above the harvested seaweed may also be conserved off-shore and subsequently the fermentation takes place under controlled conditions in a factory facility.

In an embodiment of the present invention the second combinatorial material provided in step (f), may comprise an amount of seaweed and/algae material in the range of 0.1-40% (w/w) of the second combinatorial material provided in step (f), such as in the range of 0.25-30% (w/w), e.g. in the range of 0.5-20% (w/w), such as in the range of 0.75-10% (w/w), e.g. in the range of 1-5% (w/w), such as in the range of 2-3% (w/w).

In an further embodiment of the present invention the second combinatorial material provided in step (f) may comprise more than 10% by weight dry matter (wt %) seaweed and/algae material; such as more than 15% by weight dry matter (wt %) of the second combinatorial material; e.g. more than 17 wt %; such as more than 20 wt %; e.g. more than 22 wt %; such as more than 24 wt %; e.g. more than 26 wt %; such as more than 28 wt %; e.g. more than 30 wt %; such as more than 35 wt %; e.g. more than 40 wt %; such as in the range of 10-40 wt % of the second combinatorial material may be seaweed and/or algae; e.g. in the range of 15-30 wt %; such as in the range of 18-25 wt %.

A preferred embodiment of the present invention relates to a composition comprising a fermented seaweed and/or a fermented algae; in combination with a fermented plant material, wherein the fermented plant material may be selected from the family Fabaceae.

The composition according to the present invention consists essentially of a fermented seaweed and/or a fermented algae; in combination with a fermented plant material, wherein the fermented plant material is selected from the family Fabaceae.

Preferably, the composition according to the present invention comprises a fermented seaweed and/or algae; in combination with a fermented plant material selected from the family Fabaceae and at least one lactic acid producing bacteria, as described herein.

In an embodiment of the present invention the composition does not comprise a plant material selected from the group consisting of lupine, Vicia faba (broad bean, field bean), variant of Vicia faba, such as Vicia faba var. equina (horse been), Pisum sativum, variants of Pisum sativum, such as Pisum sativum var. arvense (field pea), Medicago sativa (Alfalfa) and variant thereof.

In a further embodiment of the present invention, the composition further comprises in the range of 25-150 g lactic acid per kg fermented composition, such as in the range of 50-125 g lactic acid per kg fermented composition, such as in the range of 65-100 g lactic acid per kg fermented composition, such as about 75 g per kg fermented composition.

Preferably, the fermented composition comprises less than 15% (w/w) seaweed and/or algae, such as less than 14% (w/w), e.g. less than 13% (w/w), such as less than 12% (w/w), e.g. less than 11% (w/w), such as less than 10% (w/w), e.g. less than 9% (w/w), such as less than 7% (w/w), e.g. less than 5% (w/w), such as less than 3% (w/w).

The fibrous material from the seaweed and/or algae, and/or the plant material may be maintained in the fermented composition. Hence, the composition according to the present invention may comprises more than 5 g fibrous material originating from the starting material per kg fermented composition, such as more than 10 g fibrous material, e.g. more than 15 g fibrous material, such as more than 20 g fibrous material, e.g. more than 25 g fibrous material, such as more than 50 g fibrous material, e.g. more than 75 g fibrous material, such as more than 100 g fibrous material, e.g. more than 150 g fibrous material, such as more than 200 g fibrous material, e.g. more than 250 g fibrous material, such as more than 300 g fibrous material.

In an embodiment of the present invention the composition may further comprise a carotenoid.

Preferably, the carotenoid may be astaxanthin, β-carotene, lutein, taraxanthin, tunaxanthin, alpha-& beta-doradexanthins, zeaxanthin, or any combination hereof. Preferably, the carotenoid may comprise β-carotene and/or astaxanthin, even more preferably, the carotenoid may comprise β-carotene.

Preferably, the carotenoid present in the composition may be provided from the fermented seaweed and/or a fermented algae; in combination with a fermented plant material, wherein the fermented plant material is selected from the family Fabaceae.

In an embodiment of the present invention the composition according to the present invention comprises an amount of carotenoid, such as astaxanthin and/or β-carotene, within the range of 0.0001-5 mg/g composition, such as in the range of 0.0005-4 mg/g, e.g. in the range of 0.001-3 mg/g, such as in the range of 0.05-2.5 mg/g, e.g. in the range of 0.1-2 mg/g, such as in the range of 0.25-1.5 mg/g, e.g. in the range of 0.5-1.0 mg/g.

In a further embodiment of the present invention the composition comprises at least 0.0001 mg carotenoid, such as astaxanthin and/or β-carotene, per kg composition, such as at least 0.0005 mg/g, e.g. at least 0.001 mg/g, such as at least 0.005 mg/g, e.g. at least 0.01 mg/g, such as at least 0.05 mg/g, e.g. at least 0.1 mg/g, such as at least 0.5 mg/g, e.g. at least 1 mg/g, such as at least 1.5 mg/g, e.g. at least 2.0 mg/g, such as at least 2.5 mg/g, e.g. at least 3.0 mg/g.

Since carotenoid from may be provided as part of the fermented composition the need for adding externally produced carotenoids may be reduced or even avoided.

The externally produced carotenoids may be carotenoids not originally present in the fermented composition of the present invention and/or carotenoids that are not formed from the fermentation of the fermented seaweeds and/or algae and fermented plant material according to the present invention using lactic acid producing bacteria.

The externally produced carotenoids may be produced chemically, synthetic or by fungal fermentation.

Preferably, the amount of chemical and/or external carotenoid, in particular astaxanthin and/or β-carotene, may be below 1 mg per kg feed product, preferably fish feed product, such as below 0.5 per kg feed product, e.g. below 0.05 mg per kg feed product, such as below 0.005 per kg feed product, e.g. below 0.0005 mg per kg feed product, such as below 0.00005 mg per kg feed product, e.g. no measurable external carotenoid.

The optional drying step in the processes according to the present invention may be conducted by different means, preferably, to optimize handling, storage and viability of the composition after drying a cooling step may be introduced. In a preferred embodiment of the present invention, the drying step may be performed by a spin flash dryer.

In yet an embodiment of the present invention the fermented composition may preferably be dried by the drying method as described in DK 2011 70489.

### Food/Feed Ingredient and Food/Feed Product

The fermented composition obtained by/obtainable by the process of the present invention may form part of (or be) a food ingredient or a feed ingredient.

Thus, an aspect of the invention relates to a food ingredient or a feed ingredient comprising the fermented composition according to the invention.

In the present context “food” refers to eatable material suitable for human consumption, whereas feed refers to eatable material suitable for animal consumption. The term “animal(s)” as used herein is intended to include mammals such as pigs, piglets, cattle, and horses, poultry such as chickens, turkeys, hens, geese and ducks, and fish such as salmon and trout. Monogastric animals, such as humans, pigs, horses, dogs, and cats, have a simple single chambered stomach. In contrast, ruminant animals or ruminants have a multi-chambered complex stomach. Ruminants digests their food in two steps, first by eating the raw material and regurgitating a semi-digested form known as cud, then eating (chewing) the cud, a process called ruminating. Ruminants include for example cattle, goats, sheep and deer.

Preferably, the composition according to the present invention may be used as a feed for pigs, in particular piglets; poultry, in particular chicken; or as fish feed, in particular salmon.

In an embodiment of the present invention the composition is used as an ingredient in pig feed, in particular in feed for piglets, and wherein the ingredient constitutes in the range of 0.1-20% (w/w) of the pig feed; such as in the range of 1.0-15% (w/w); e.g. in the range of 2.0-12% (w/w); such as in the range of 3.0-11% (w/w); e.g. in the range of 4.0-10% (w/w); such as in the range of 5-9% (w/w); e.g. in the range of 6.0-8.0% (w/w); such as about 7% (w/w).

The composition according to the present invention may be used in a poultry feed. The poultry feed, in particular the chicken feed, may comprise the composition according to the present invention. Preferably, the poultry feed comprises in the range of 0.1-20% (w/w) of the chicken feed; such as in the range of 0.25-15% (w/w); e.g. in the range of 0.5-10% (w/w); such as in the range of 0.75-8% (w/w); e.g. in the range of 1.0-7% (w/w); such as in the range of 1.5-6% (w/w); e.g. in the range of 2.0-5% (w/w); such as in the range of 2.5-4% (w/w); e.g. about 3% (w/w).

The composition according to the present invention may be used in a fish feed. The fish feed, in particular the salmonoid fish feed, may comprise the composition according to the present invention.

In an embodiment of the present invention the composition according to the present invention constitutes in the range of 1-30% (w/w) of the fish feed; such as in the range of 2.5-28% (w/w); e.g. in the range of 5-25% (w/w); such as in the range of 7.5-23% (w/w); e.g. in the range of 10-20% (w/w); such as in the range of 12-18% (w/w); e.g. in the range of 14-16% (w/w); such as about 15% (w/w).

Market analysis have shown that the stronger the colour of salmonoid the more attractive it is for the customers, and thus may be preferred.

The fish feed according to the present invention may preferably be a salmonoid fish feed. The salmonoid may include salmon (both ocean-going and lake-locked), trout, chars, freshwater whitefishes, and graylings.

Preferably, the salmonoid may be a salmon or a trout.

A preferred embodiment of the present invention relates to a composition comprising a fermented seaweed and/or a fermented algae (a fermented seaweed; a fermented algae or a combination of a fermented seaweed and a fermented algae); in combination with a fermented plant material, wherein the fermented plant material is selected from the family Fabaceae, preferably Glycine mas (soybean), wherein the composition, when may be fed to a salmonoid, to obtain a colour intensity above 25 when measured by the SalmoFan™ colour measurement scale.

The colour intensity when measured by the SalmoFan™ colour measurement scale may preferably be above 26; such as above 27; e.g. above 28; such as above 29; e.g. above 30; such as above 31; e.g. above 32.

Preferably, the colour intensity of a salmonoid filet obtained from a salmonoid fed a composition according to the present invention, may be improved (increased) by at least 5%, relative to the colour intensity of the salmonoid filet obtained from a salmonoid fed with fungal or chemical produced carotenoid, in particular astaxanthin and/or β-carotene, such as at least 10%, e.g. at least 15%, such as at least 20%, e.g. at least 25%.

Preferably, the colour intensity of a salmonoid filet obtained from a salmonoid fed a composition according to the present invention, may be improved (increased) by at least 2%, relative to the colour intensity of the salmonoid filet obtained from a salmonoid fed with a composition comprising a combination of fermented rapeseed meal and fermented seaweed, such as at least 3%, e.g. at least 4%, such as at least 5%, e.g. at least 6%, such as at least 7%, e.g. at least 8%, such as at least 9%, e.g. at least 10%.

It is generally accepted that the colour of e.g. salmon fillets is one of the most important quality parameters when a customer is shopping salmon. Therefore, colour of salmonoids is a key element when farming salmonoids and plays a decisive role when evaluating the quality of the product at the point-of-sale.

In conventional Norwegian salmon farming, the cost of carotenoid accounts for approximately 15% of the feed costs. Furthermore, the feed costs account for approximately 50% of the total production costs for farming salmonoids. Hence, colouring of the fillets and the addition of carotenoid to the feed may be a relatively significant cost in salmon farming.

The colour intensity of a salmonoid fillets according to the present invention may be determined by the SalmoFan™ color measurement scale. The SalmoFan™ color measurement scale has been developed by DSM and may be determined physically, using visual inspection applying a colour scale, or by digitally determination, using a colour sensor to determine the colour intensity.

In an embodiment of the present invention the composition according to the present invention comprises a range of 30-70% (w/w); such as a range of 40-60% (w/w); e.g. about 50% (w/w) of the composition comprises a particle size below 0.5 mm and a range of 30-70% (w/w); such as a range of 40-60% (w/w); e.g. about 50% (w/w) of the composition comprises a particle size above 0.5 mm.

Preferably, in the range of 20-80% (w/w) of the composition according to the present invention comprises a particle size below 0.5 mm (preferably in the range of 0.01-0.5 mm), and wherein in the range of 20-80% (w/w) of the composition according to the present invention comprises a particle size above 0.5 mm (preferably in the range of 0.5-1.0 mm)

In an even further embodiment of the present invention the composition according to the present invention comprises at least 2, preferably at least 3, even more preferably at least 4 of the following criteria:
a) 1-10% (w/w), such as about 5% (w/w), of the composition comprises a particle size above 1.0 mm;
b) 45-55% (w/w), such as about 50% (w/w), of the composition comprises a particle size between 0.5-1.0 mm;
c) 30-40% (w/w), such as about 50% (w/w), of the composition comprises a particle size between 0.25-0.5 mm; and/or
d) 5-15% (w/w), such as about 10% (w/w), of the composition comprises a particle size below 0.25 mm.

In the present context the term “about” relates to a variation on the stated amount of 10% or less, such as 5% or less, e.g. 1% or less.

The selection of the various particle sizes may be determined by sieving as known to the skilled person.

In a further embodiment of the present invention the composition may be a dried composition (e.g. a fermented dried composition).

The food and/or feed ingredient may also form part of a food/feed product. Thus, in a further aspect the invention relates to a food product and/or a feed product comprising the food and/or feed ingredient according to the present invention.

A preferred embodiment of the present invention relates to the use of the fermented composition according to the present invention for:
increasing the colour in a salmonoid;
increased growth of fish and/or animals;
reducing mortality;
increased feed conversion; and/or
reduced feed consumption of fish and/or animals.

In yet a further aspect the fermented composition is a pre-composition for use in the production of biofuel, such as bio-ethanol.

An additional aspect relates to the use of the fermented composition according to the invention as a food/feed ingredient.

In an embodiment of the present invention the concentration of viable lactic acid producing bacteria in inoculum provided in step (a) may be above 10⁷ CFU per gram; such as above 10⁸ CFU per gram; e.g. above 10⁹ CFU per gram; such as in the range 10⁷ to 10¹² CFU per gram; e.g. in the range 10⁸ to 10¹¹ CFU per gram; such as in the range 10⁹ to 10¹⁰ CFU per gram.

It should be noted that embodiments and features described in the context of one of the aspects or embodiments of the present invention also apply to the other aspects or embodiments of the invention. All patent and non-patent references cited in the present application, are hereby incorporated by reference in their entirety.

### EXAMPLES

### Example 1

A method according to the present invention for providing a fermented composition according to the present invention comprising a combination of 1% seaweed (50% (w/w) Laminaria saccharina and 50% (w/w) Ascophyllum nodosum) and a plant material comprising 93% Glycine max (soybean) meal and 6% rapeseed meal.

Seaweed (Laminaria saccharina/Ascophyllum nodosum) was pre-treated to reduce the average diameter in a chopper, and mixed with a combination of the lactic acid-producing bacteria Pediococcus pentosaceus, Pediococcus acidilactici and Lactobacillus plantarum in a fermentation tank and the moisture content was adjusted to about 40% humidity. The oxygen present in the fermentation tank was removed by vacuum and the fermentation was performed at a temperature of about 30° C.

After 3 days of fermentation of the seaweed, a plant material comprising a combination of soybean meal and rapeseed meal, which was also reduced in size, was added to the fermented seaweed, resulting in a composition comprising 1% (w/w) seaweed, 93% (w/w) soybean meal, and 6% (w/w) rapeseed meal in a fermentation tank; oxygen present in the fermentation tank was removed by vacuum and the fermentation was continued for 3 days at 25° C.

The resulting fermented composition had a bacterial count of 3.2×10⁶ CFU/gram, a pH of 4.1 and a lactic acid concentration of about 75 g lactic acid per kg composition.

The resulting fermented composition comprising the combination of seaweed, plant material and lactic acid bacteria, was subjected to spin flash drying to provide a dried fermented composition where the lactic acid bacteria were protected and a high number of viable cells was kept.

About 40% (w/w) of the dried fermented composition comprises a particle size below 0.5 mm (mainly in the range of 0.1-0.5 mm) and about 60% (w/w) of the composition comprises a particle size above 0.5 mm (mainly in the range of 0.5-1.0 mm).

### Example 2.

Fish feeding experiment using the following feed are performed:
(1) A basic fish feed comprising 50 mg chemically produced astaxanthin/kg basic fish feed;
(2) A basic fish feed comprising 15% (w/w) of a fermented composition comprising 99% fermented rapeseed meal and 1% fermented seaweed; and
(3) A basic fish feed comprising 15% (w/w) of a fermented composition as provided in Example 1, comprising 93% fermented soymeal, 6% fermented rapeseed meal and 1% fermented seaweed.

3 ponds with each 10 salmons of about 3 kg per salmon was fed with the above-mentioned basic fish feed, including supplement (1), (2) or (3), in the stated concentrations, for a period of 8 weeks. After the four weeks the salmons were harvested, gutted and filleted and the colour of the fillets was determined by the SalmoFan™ color measurement scale.

From the SalmoFan™ color measurement scale the following results was obtained:

| Supplement used in the basic fish feed | Colour intensity |
|---|---|
| Chemical astaxanthin (supplement 1) | 23-23 |
| Mixture of fermented rapeseed meal | 23-24 |
| and seaweed (supplement 2) | |
| Mixture of fermented soy meal, rape | 29-30 |
| seed meal and seaweed | |
| (supplement 3) | |

Thus, the fillets obtained from salmons fed with basic fish feed comprising 15% (w/w) of the basis fish feed of fermented composition comprising 93% fermented soymeal, 6% fermented rapeseed meal, and 1% fermented seaweed, as provided in Example 1, showed to have a significantly stronger colour intensity (higher value on the SalmoFan™ color measurement scale) relative to the salmon fillets obtained from feeding fish with commercial feed comprising chemically produced astaxanthin and fish feed comprising fermented rapeseed meal and seaweed.

### Example 3.

A feeding experiment for chicken using the following feed:
(1) A basis feed (without any supplementation);
(2) A basic feed comprising 5% (w/w) fermented rapemeal;
(3) A basic feed comprising 5% (w/w) fermented composition comprising 80% fermented rapemeal and 20% fermented seaweed; and
(4) A basic feed comprising 5% (w/w) fermented composition as provided in Example 1, comprising 93% fermented soymeal, 6% rapeseed meal and 1% fermented seaweed.

The feeding using the supplemented feeds described in feed (2)-(4) was performed for a first period of 2 weeks followed by a second period of 2 weeks where the content of the fermented composition was reduced to 2% (w/w). Following the second period of feeding no supplementation of fermented composition was done.

The following table shows the effect growth of chicken fed a feed comprising a composition according to the present invention, and as provided according to Example 1.

| Feed | ABW-0 (kg) | AWB-13 (kg) | AWB-20 (kg) |
|---|---|---|---|
| (1) | 0.043 | 0.486 | 1.056 |
| (2) | 0.043 | 0.494 | 1.084 |
| (3) | 0.044 | 0.491 | 1.071 |
| (4) | 0.043 | 0.511 | 1.117 |

ABW-0 = Average Body Weight after 0 days;
ABW-13 = Average Body Weight after 13 days;
ABW-20 = Average Body Weight after 20 days;
ABW-35 = Average Body Weight after 35 days.

The results show that the daily weight gain as well as the feed conversion for chicken receiving the fourth feed (basic feed comprising 5% (w/w) fermented composition as provided in Example 1), are much more effective and outperforms other feeds.

The major difference may be observed until 20 days of age. After the 20 days of 5 age, the difference may become smaller, but stays significantly more effective (data not shown).

1. A composition comprising a fermented seaweed and/or a fermented algae; in combination with a fermented plant material, wherein the fermented plant material is selected from the family Fabaceae.

2. The composition according to claim 1, wherein the composition further comprises in the range of 25-150 g lactic acid per kg fermented composition.

3. The composition according to claim 1, wherein the composition comprises less than 15% (w/w) seaweed and/or algae, such as less than 14% (w/w), e.g. less than 13% (w/w), such as less than 12% (w/w), e.g. less than 11% (w/w), such as less than 10% (w/w), e.g. less than 9% (w/w), such as less than 7% (w/w), e.g. less than 5% (w/w), such as less than 3% (w/w).

4. The composition according to claim 1, wherein the composition further comprises carotenoid.

5. The composition according to claim 4, wherein the composition comprises at least 0.0001 g carotenoid, such as astaxanthin and/or β-carotene, in particular β-carotene, per kg composition.

6. A process for providing a composition comprising the combination of fermented seaweeds and/or algae and fermented plant material, wherein the plant material is Glycine max (soybean), the process comprises:
a) providing an inoculum comprising lactic acid-producing bacteria;
b) providing a first material to be fermented, where said first material comprises seaweeds and/or algae;
c) optionally, providing a source of enzyme, such as phosphatases e.g. phytase;
d) combining the materials of steps a), (b) and optionally c), thereby providing a first combinatorial material;
e) fermenting the first combinatorial material of step d) using the inoculum of step a) at a temperature in the range of 15-45° C. and for a period in the range 2-40 days; thereby providing a first fermented composition;
f) combining the first fermented composition of step e) with a second material to be fermented, wherein said second material comprises a plant material selected from, wherein the plant material is Glycine max (soybean), thereby providing a second combinatorial material;
g) fermenting the second combinatorial material of step f), using the first fermented composition of step e) as a inoculum, at a temperature in the range of 15-45° C. and for a period of at least 2 days, such as at least 3 days, e.g. at least 4 days, such as at least 5 days, such as in the range of 2-15 days, e.g. in the range of 5-12 days, such as 5-10 days, such as 5-7 days or such as 8-10 days; thereby providing a second fermented composition comprising fermented seaweeds and/or algae and fermented plant material, wherein the fermented plant material is fermented Glycine max (soybean); and
h) optionally, drying the second fermented composition.

7. A process for providing a composition comprising the combination of fermented seaweeds and/or algae and fermented plant material, wherein the plant material is Glycine max (soybean), the process comprises:
a) providing an inoculum consist essentially of lactic acid-producing bacteria;
b) providing a first material to be fermented, where said first material consist essentially of seaweeds and/or algae;
c) optionally, providing a source of enzyme, such as phosphatases e.g. phytase;
d) combining the materials of steps a), (b) and optionally c), thereby providing a first combinatorial material;
e) fermenting the first combinatorial material of step d) using the inoculum of step a) at a temperature in the range of 15-45° C. and for a period in the range 2-40 days; thereby providing a first fermented composition;
f) combining the first fermented composition of step e) with a second material to be fermented, wherein said second material consist essentially of a plant material, wherein the plant material is Glycine max (soybean), thereby providing a second combinatorial material;
g) fermenting the second combinatorial material of step f), using the first fermented composition of step e) as a inoculum, at a temperature in the range of 15-45° C. and for a period of at least 2 days, such as at least 3 days, e.g. at least 4 days, such as at least 5 days, such as in the range of 2-15 days, e.g. in the range of 5-12 days, such as 5-10 days, such as 5-7 days or such as 8-10 days; thereby providing a second fermented composition comprising fermented seaweeds and/or algae and fermented plant material, wherein the fermented plant material is fermented Glycine max (soybean); and
h) optionally, drying the second fermented composition.

8. The process according to claim 6, wherein a further inoculum comprising lactic acid-producing bacteria is added to the second combinational material provided in step (f)

9. The process according to claim 6, wherein the lactic acid-producing bacteria in the inoculum used for fermentation are lactic acid bacteria of the genus Enterococcus, Lactobacillus, Pediococcus or Lactococcus, or combinations thereof.

10. The process according to claim 9, wherein neither the seaweeds; the algae or the plant material, is subjected to a sterilization step.

11. The process according to claim 6, wherein the fermentation of the first combinatorial material in step e) is performed at a temperature in the range 15-45° C., such as 15-40° C., such as 25-35° C., such as 30-40°° C., such as 15-20° C. or such as 40-45° C. and wherein the fermentation of the first combinatorial material in step e) is performed for a period in the range of 3-8 days, such as 3-5 days, or such as 5-10 days and wherein the fermentation of the second combinatorial material in step f) is performed at a temperature in the range 15-45° C., such as 15-40° C., such as 25-35° C., such as 30-40° C., such as 15-20° C. or such as 40-45°° C. and wherein the fermentation of the second combinatorial material in step f) is performed for a period in the range of 3-8 days, such as 3-5 days, or such as 5-10 days.

12. A fermented composition obtained/obtainable by the process according to claim 6.

13. A feed ingredient comprising the fermented composition according to claim 1.

14. A food/feed product comprising the food/feed ingredient according to claim 13.

15. Use of the fermented composition according to claim 1 or
increasing the colour in a salmonoid;
increased growth of fish and/or animals;
reducing mortality;
increased feed conversion; and/or
reduced feed consumption of fish and/or animals.
